(19) United States
(12) Patent Application Publication
(10) Pub. No.: US 2007/0287995 A1
(43) Pub. Date: Dec. 13, 2007

(54) Cooled Ablation Catheter and Method of Using the Same

(75) Inventor: Martin L. Mayse, St. Louis, MO (US)

Correspondence Address: SENNIGER POWERS, ONE METROPOLITAN SQUARE, 16TH FLOOR, ST LOUIS, MO, 63102, US

(73) Assignee: WASHINGTON UNIVERSITY, St. Louis, MO (US)

(21) Appl. No.: 11/422,912

(22) Filed: Jun. 8, 2006

(51) Int. Cl. A61B 18/14

(52) U.S. Cl. 606/41

(57) ABSTRACT

A cooled high frequency energy ablation catheter uses an expansion of a fluid inside the catheter near a tip to cool the tip. The tip is brought into contact with a contact region of the tissue to deliver high frequency energy to the contact region and underlying tissue. The contact region of the tissue is maintained at a temperature less than 50° C. (122° F.) using the cooled, expanded fluid to prevent charring of the contact region. The tissue underlying the contact region is heated by resistive heating to a temperature of greater than 50° C. (122° F.) to ablate the underlying tissue.

### BACKGROUND OF THE INVENTION

In general, the present invention relates to an ablation catheter, and more particularly, to an ablation catheter having a tip that can be cooled.

Catheters in general have a broad range of applications in the medical field. One type of catheter is an ablation catheter. An ablation catheter is typically used to destroy unwanted tissue within the human body by using, for example, radio frequency energy (radio frequency ablation) or extreme cold (cryoablation). For example, for patients with an abnormal heart rhythm, an ablation catheter (referred to as a cardiac ablation catheter) may be used to destroy heart tissue that creates abnormal electrical signals. The catheter is fed through a heart vessel and into or onto the heart where the ablation takes place. Other types of ablation catheters may be used in the treatment of obstructed airway tumors in the lung, pulmonary nodules in the lung, and bladder cancer.

A conventional radio frequency ablation catheter typically includes a conducting electrode, typically a tip of the catheter, connected to a supply of radio frequency energy. Radio frequency energy is applied to the tissue via the conducting electrode, which is placed in contact with the tissue. The relatively high electrical impedance at the electrode-tissue interface results in local heating and thermal tissue destruction.

Conventional radio frequency ablation catheters have a major drawback because uncontrolled heating of the tissue limits the depth of tissue destruction. Uncontrolled heating “chars” the tissue at the electrode-tissue interface and effectively lowers the impedance of the tissue at the interface, severely limiting further heating of the tissue beneath the charred tissue. To overcome this drawback, conventional catheters cool the tip of the catheter by running cool saline, typically from a saline bath, within the catheter adjacent to the tip. Excess heat transfers from the electrode to the saline to cool the tip to inhibit charring, while the radio frequency heating is maintained beyond the tissue-electrode interface. The saline may be either continuously circulated between the saline bath and the catheter tip or it may be discharged from the catheter after cooling the catheter.

To properly cool the tip, the passage carrying the saline must be large enough to allow for an adequate flow rate of the saline within. Therefore, the minimum diameter of the saline cooled catheter is restricted. Moreover, for use with steerable catheters, the large saline passage may interfere with the steerability and flexibility of the catheter. Further, the tip of a saline cooled catheter typically cannot be cooled to a temperature below 10 to 15° C. (50 to 59° F.) because the saline liquid is heated as it passes through the catheter towards the tip via a counter-current heat exchange. This limitation restricts the amount of heat that can be pulled from the tip of the catheter over a short period of time. Moreover, the use of a saline bath is rather cumbersome for the user.

### SUMMARY OF THE INVENTION

In one aspect of the present invention, a method of ablating tissue using a cooled high frequency ablation catheter generally comprises supplying high frequency energy to the electrically conductive tip of the catheter. A compressed fluid is expanded in an expansion chamber of the catheter to endothermically cool the fluid. The electrically conductive tip of the catheter is cooled using the expanding fluid. The fluid is exhausted from the expansion chamber. The tip of the catheter is brought into contact with a contact region of the tissue to deliver high frequency energy to the contact region and underlying tissue. The contact region is maintained at a temperature between about 5° C. (41° F.) and about −5° C. (23° F.) to prevent charring of the contact region. The tissue underlying the contact region is heated by resistive heating to a temperature of greater than about 50° C. (122° F.) to ablate the underlying tissue.

In another aspect of the present invention, a method of ablating tissue using a high frequency ablation catheter generally comprises supplying an electrically conductive tip of the ablation catheter with high frequency energy. The tip of the ablation catheter contacts the tissue. The tip of the ablation catheter is cooled to a temperature between about 5° C. (41° F.) and about −5° C. (23° F.).

In yet another aspect of the present invention, a high frequency energy ablation catheter having a proximal end and a distal end generally comprises a first passage having an inlet generally at the proximal end of the catheter and an outlet generally at the distal end of the catheter. The first passage is adapted to deliver pressurized fluid from the inlet through the outlet. An expansion valve is disposed at the outlet of the first passage and is adapted to restrict flow out of the first passage to maintain pressure of the fluid within the first passage. An expansion chamber is in fluid communication with the expansion valve, and the pressure within the expansion chamber is less than the pressure inside the first passage. An electrically conductive tip adjacent to the expansion valve at the distal end of the catheter is connected to a source of high frequency energy for delivering the high frequency energy to tissue. The catheter is adapted to deliver the high frequency energy generally axially away from the distal and proximal ends of the catheter when the tip is in contact with the tissue. Pressurized fluid that enters into the first passage passes through the expansion valve and expands endothermically into the expansion chamber, thereby cooling the tip of the catheter.

Other objects and features will be in part apparent and in part pointed out hereinafter.

### BRIEF DESCRIPTION OF THE DRAWINGS

FIG. 1 is a side elevation of one embodiment of a catheter of the present invention;

FIG. 2 is an enlarged fragmentary section of a body of the catheter of FIG. 1;

FIG. 3 is an enlarged section of a distal portion of the catheter;

FIG. 4 is a cross-section of the catheter taken in the plane including the line 4-4 of FIG. 1;

FIG. 5 is a cross-section of the catheter taken in the plane including the line 5-5 of FIG. 1

Corresponding reference characters indicate corresponding parts throughout the drawings.

### DETAILED DESCRIPTION OF THE DRAWINGS

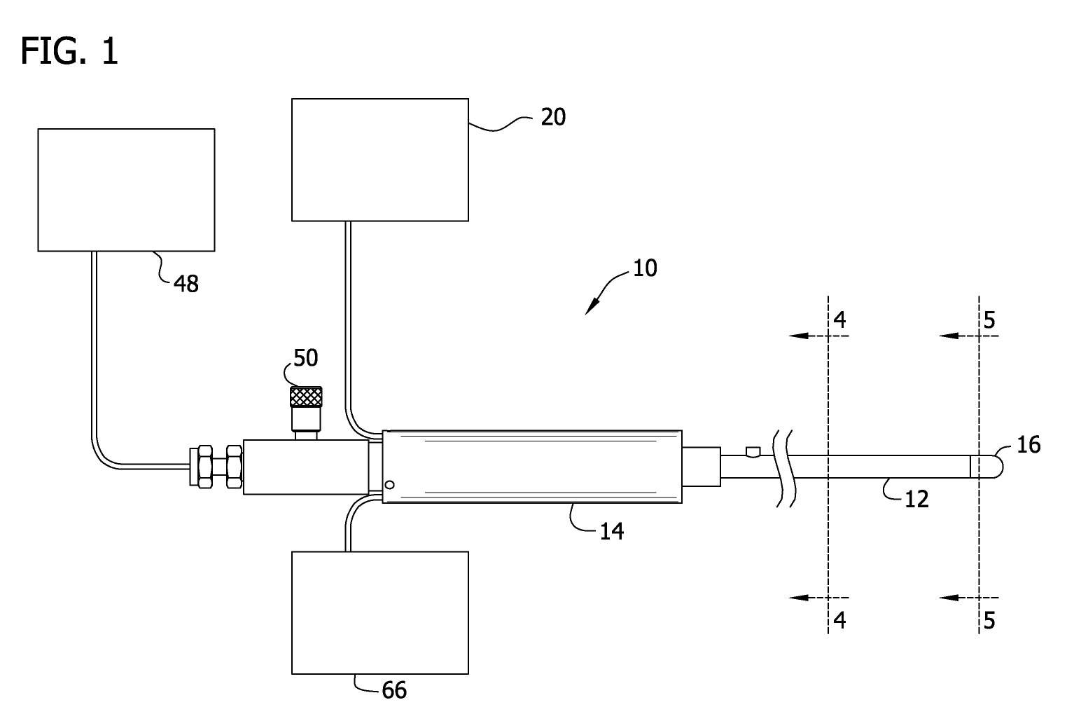

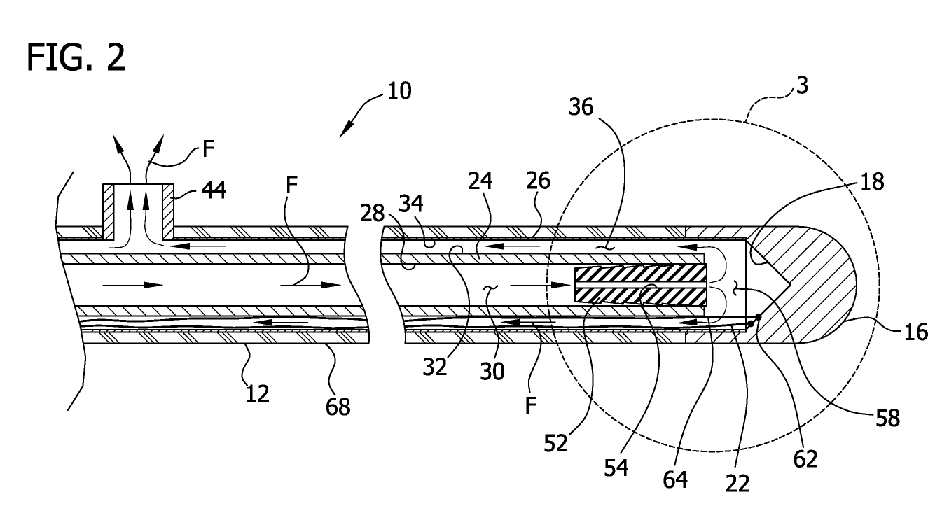

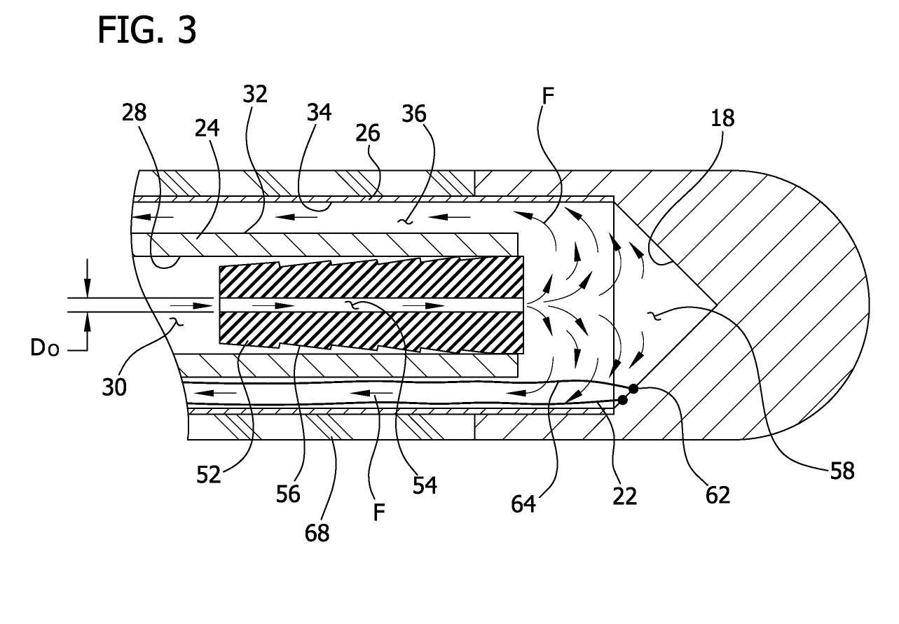

Referring now to the Figures, and in particular to FIGS. 1-3, an ablation catheter of the present invention is generally indicated at 10. The ablation catheter 10 comprises an elongate body 12 having a handle 14 disposed at a proximal end and a tip 16 disposed at a distal end. It is understood that the term “proximal” refers to a portion of the catheter closer to an operator, and the term “distal” refers to a portion of the catheter 10 farther away from the operator. As shown in FIGS. 2 and 3, the distal end of the body 12 is snugly received in a cavity 18 of the tip 16 and adhesive or other material is used to secure the tip to the body. Other ways of attaching the tip 16 to the body 12 are within the scope of this invention. Although the tip 16 is formed separately from the body 12 in the illustrated embodiment, it is contemplated that the body and tip may be formed as one piece of material.

The tip 16 is electrically connected to a source of radio frequency energy 20 (broadly, a source of high frequency energy) via an electrically conductive element 22 (e.g., a wire) (FIGS. 2 and 3) running lengthwise between the ends of the body 12. The ablation catheter 10 introduces radio frequency energy into tissue via the tip 16 when the tip contacts the tissue. When the energy encounters the high impedance of tissue, the tissue is heated through resistive (also referred to as ohmic) heating, causing thermal injury and death of the tissue. The tip 16 is constructed of metal, such as stainless steel, although it may be constructed of other electrically conductive material.

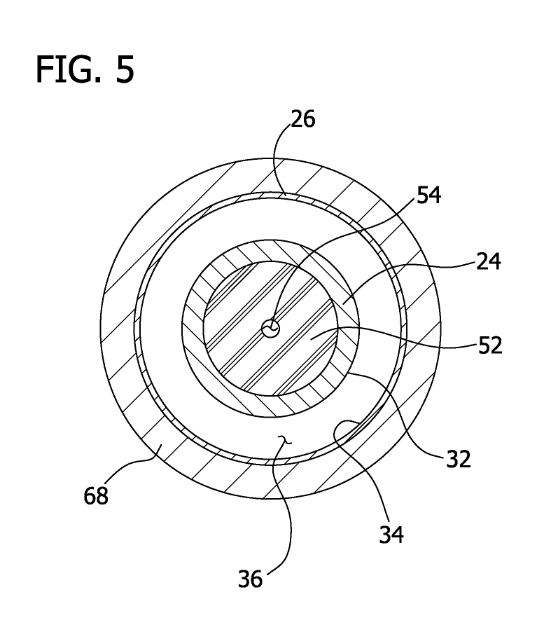

Referring to FIGS. 2-5, the body 12 of the illustrated catheter 10 comprises an inner tubular member 24 received in an outer tubular member 26. An interior surface 28 of the inner tubular member 24 defines a first passage 30 (i.e., an inner passage) within the body 12, and an exterior surface 32 of the inner tubular member and an interior surface 34 of the outer tubular member 26 define a coaxial second passage 36 (i.e., an outer passage) surrounding the first passage. Alternatively, it is understood that the passages 30, 36 may not be coaxial and/or may be disposed side by side within the body 12. The first passage 30 has an inlet adjacent the proximal end of the body 12 and an outlet adjacent the distal end of the body and the tip 16. The second passage 36 has an inlet adjacent the distal end of the body 12 and an outlet 44 (FIG. 2) generally adjacent the proximal end of the body. The body 12 may be constructed in other ways without departing from the scope of the invention. For example, the body 12 may be of a one-piece construction, such as rod stock, and the passages 30, 36 may be formed by boring. Alternatively, the body 12, including the passages 30, 36, may be formed by molding. Other ways of constructing the catheter 10 are within the scope of this invention.

The inlet of the first passage 30 is fluidly connected to a source 48 (FIG. 1) of compressed fluid, indicated by arrows F, such as a compressed gas. As explained in detail below, the tip 16 of the catheter 10 is cooled by expanding the compressed fluid F in the catheter. Suitable fluids F for use with this invention include, but are not limited to, nitrous oxide, and carbon dioxide. For example, when the fluid is nitrous oxide, the source 48 of fluid F may be pressurized to about 5267.6 kPa (52 atm). The preferred range of pressure may be other than described. For example, the pressure is dependant upon the type of fluid F that is used and the ambient temperature at which the fluid F is utilized. It is understood that because the fluid F is highly pressurized at the source 48, it may be in the form of a gas or a liquid at the source. A control valve 50 (FIG. 1) disposed between the catheter 10 and the source 48 of compressed fluid F regulates the flow of the fluid into the first passage 30. The control valve 50 may be part of the source 48 of compressed fluid F, part of the catheter 10, or may be independent of both as long as it is in fluid communication with the source of fluid and the first passage 30.

The outlet of the first passage 30 includes an expansion valve 52 for retarding the flow of fluid F out of the first passage 30, thereby maintaining the fluid at a relatively high pressure within the first passage. Because the fluid F is maintained at such a high pressure, it is understood that the fluid within the first passage 30 may be either in the form of gas or liquid or both. For example, when the fluid F is nitrous oxide, the pressure of the fluid F within the first passage 30 is between about 304.0 kPa (3 atm) and about 5267.6 kPa (52 atm). The range of pressure may be other than described. For example, the pressure range is dependent upon the type of fluid F that is used and the ambient temperature at which the fluid F is utilized. The expansion valve 52 tapers toward its proximal end and comprises ridges 56 formed on its outer surface to aid in retaining the valve within the first passage 30 against the force exerted upon it by the pressurized fluid F. Other ways of constructing the expansion valve 52 are contemplated and within the scope of this invention. For example, the first passage 30 and the expansion valve 52 may be of a one-piece construction.

Referring to FIGS. 2, 3 and 5, the expansion valve 52 has an orifice 54 extending longitudinally therethrough. The orifice 54 has a cross-sectional diameter Dₒ (FIG. 3) less than the cross-sectional diameter of the first passage 30. The cross-sectional diameter Dₒ of the orifice 54 should be sufficient to retard the flow of the pressurized fluid F from the fluid source 48 enough to maintain the fluid in the first passage 30 at the desired pressure. Preferably, the diameter Dₒ of the orifice 54 is between about 0.015 inches (0.038 cm) and about 0.005 inches (0.013 cm), although the orifice may be other sizes depending on the type of fluid used and the desired pressure of the fluid. Although the expansion valve 52 illustrated is of fixed dimensions, an adjustable orifice expansion valve may be used.

An expansion chamber 58 (FIGS. 2 and 3) within the body 12 disposed adjacent the tip 16 is in fluid communication with the expansion valve 52. Pressure within the expansion chamber 58 is less than the pressure within the first passage 30. For example, the pressure may be slightly more than atmospheric pressure, such as between about 101.3 kPa (1 atm) and about 202.6 kPa (2 atm). As the high pressure fluid F within the first passage 30 enters the relatively low pressure in the expansion chamber 58, the fluid expands endothermically, thereby dropping its temperature rapidly. This phenomenon is generally referred to as the Joule-Thomson effect. In some situations, for example, where the fluid F is a gas in the form of a liquid within the orifice 54 of the expansion valve 52, the fluid changes into a gas as it enters the expansion chamber 58 because of the low pressure. Phase-changing also has the effect of lowering the temperature of the fluid F, as expanding from a liquid to a gas is an endothermic reaction.

In one example, when using nitrous oxide as the compressed fluid, the temperature of the fluid, after expansion, may be between about 0° C. (32° F.) and −89° C. (−128° F.). The preferred temperature range may be other than described. For example, the temperature is dependent upon the type of fluid F that is used. The decrease in temperature is transferred to the tip 16 of the catheter 10, thereby rapidly cooling it.

Cooling the tip 16 of the catheter 10 prevents overheating and “charring” of tissue that is in direct contact with the tip (e.g., the contact region of the tissue), thereby allowing more high frequency energy to penetrate deeper into the tissue and more tissue to be ablated. The desired temperature of the tip 16 to maintain the temperature of the tissue at the contact region will depend generally on the amount of energy being supplied to the tip. In one example, when using nitrous oxide, the tip 16 of the catheter 10 may be cooled to between about 5° C. (41° F.) and about −5° C. (23° F.) by the cooled fluid F, depending upon the amount of power being supplied to the tip by the source of high frequency energy. In this example, the cooled tip 16 preferably removes heat from the tissue in contact with the tip at a rate of between about 15 Watts (J/s) and 25 Watts (J/s), although higher rates of heat removal are possible. Other temperature ranges and heat removal rates are within the scope of this invention.

A thermocouple 62 (FIGS. 2 and 3) mounted adjacent the tip 16 detects the temperature of the tip. Leads 64 extend from the thermocouple 62 through the body 12 of the catheter 10 to the proximal end thereof. The leads 64 are connected to a suitable device (such as a microprocessor) for reading the temperature at the tip 16. The thermocouple 62, the control valve 50 and the source of radio frequency energy may be integrated such that a controller 66 (such as a microprocessor) regulates the temperature of the tip 16 by reading the temperature of the tip using the thermocouple 62 and controlling the supply of the pressurized fluid F and/or the radio frequency energy. For example, if the tip 16 is too hot (e.g., above 50° C. (122° F.)), the controller 66 will increase the flow of fluid F into the first passage 30, thereby increasing the amount of fluid expanding in the expansion chamber 58. Alternatively, the amounts of pressurized fluid and radio frequency energy may be controlled manually by the physician or surgeon.

In the illustrated embodiment, electrical and thermal insulation material 68, such as heat shrink material or polyurethane, surrounds the outer surface of the body 12 for electrically insulating the body 12 from the tip 16 to ensure that the radio frequency energy is supplied only to the tip and for thermally insulating the body from the tip to aid in inhibiting thermal energy transferring from the tip to the body.

The inlet of the second passage 36 fluidly communicates with the expansion chamber 58, and the outlet 44 of the second passage 36 fluidly communicates with a volume outside the body 12 of catheter 10 at a pressure (e.g., atmospheric pressure) less than the pressure inside the expansion chamber 58. For example, the outlet 44 may be open to the atmosphere. As described above, the outlet 44 is adjacent the proximal end of the body 12. This arrangement ensures that the fluid F does not enter adjacent tissue of the patient. It is contemplated that the outlet 44 may be adjacent the distal end of the body 12, particularly if the exiting fluid F is not harmful to the patient.

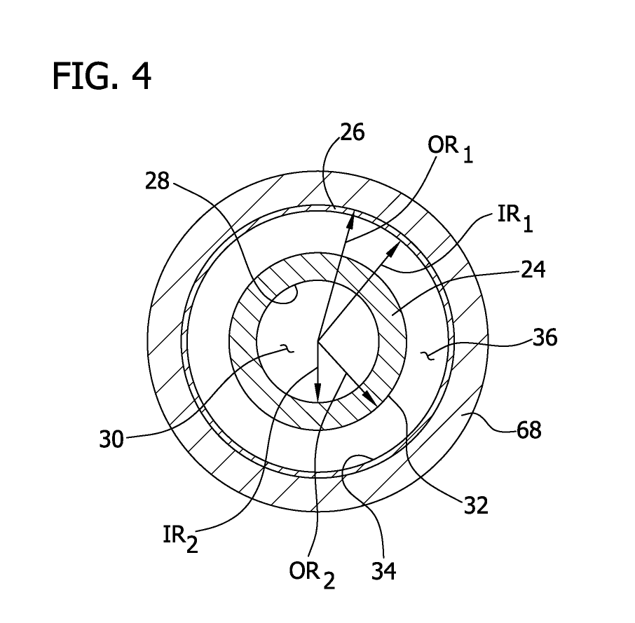

In one example, referring to FIG. 4, the outer tubular member 26 has an outer cross-sectional radius OR₁ of between about 0.0750 inches (0.019 cm) and about 0.025 inches (0.064 cm) and an inner cross-sectional radius IR₁ of between about 0.055 inches (0.133 cm) and about 0.019 inches (0.048 cm), and the inner tubular member 24 has an outer cross-sectional radius OR₂ of between about 0.041 inches (0.104 cm) and about 0.013 inches (0.033 cm) and an inner cross-sectional radius IR₂ (i.e., radius of the first passage) of between about 0.031 inches (0.078 cm) and about 0.010 inches (0.025 cm). The first passage 30 has a cross-sectional area between about 0.0030 in² (0.019 cm²) and about 0.00031 in² (0.0019 cm²). The second passage 36 has a cross-sectional area between about 0.0042 in² (0.027 cm²) and about 0.00060 in² (0.0039 cm²). Other shapes and sizes of the tubular members 24, 26 are within the scope of the invention.

In one example suitable for a rigid catheter for use in ablating pulmonary nodules, for example, the inner and outer tubular members 24, 26, respectively, are constructed of rigid material. For example, the inner tubular 24 member may be constructed of a polymer material, such as polyetheretherketone (PEEK®) and polytetrafluoroethylene (Teflon®), and the outer tubular member 26 may be constructed the same polymer material or metal, such as stainless steel.

Alternatively, in another example, the catheter 10 may be flexible and include guide wires (not shown) running longitudinally within the outer tubular member 26 for use in controllably moving the distal end of the catheter 10 and the tip 16. The guide wires and the means by which the wires move the body 12 are conventional and will not be described in detail herein. As stated above, the fluid F is a gas, the diameters of the passages 30, 36 and the diameters of the tubular members 24, 26 may be smaller than conventional saline cooled catheters. Smaller diameter passages 30, 36 and tubular members 24, 26 allow for more steerability of the catheter 10. Moreover, the overall diameter of the catheter 10 may be smaller than conventional saline cooled catheters because of the smaller diameter passages 30, 36. These aspects of one example of the present invention will be appreciated by those skilled in the art.

In use, the tip 16 of the ablation catheter 10 is positioned into contact with the tissue to be ablated at the contact region. Radio frequency energy is supplied to the tip (e.g., by turning on the source 20 of radio frequency energy) through the electrically conductive element 22. The radio frequency energy enters the associated tissue via the tip 16, and the tissue is heated by resistive heating. Simultaneously, or prior to supplying the radio frequency energy, the control valve 50 is opened. The fluid F from the compressed source 48 flows through first passage 30 and enters the orifice 54 of the expansion valve 52. As the fluid F exits the expansion valve 52, it expands, thereby undergoing the Joule-Thomson effect, whereby the temperature of the fluid decreases rapidly. The cooled fluid F cools the tip 16 of the catheter 10. The temperature of the tip 16 is controlled using the thermocouple 62 and controller 66, as described above, such that the tissue that is in contact with the tip does not exceed a temperature above about 50° C. (122° F.), the temperature at which tissue normally “chars”. The temperature of the tip 16 may be between about 5° C. (23° F.) and about −5° C. (41° F.). Using this method, the tissue at the contact region does not overheat or char, and more energy is transferred to the underlying tissue.

In one example, the fluid is provided into the catheter 10 and the tip 16 is cooled by the expanding fluid before the radio frequency energy is supplied to the tip. The tip is cooled to a temperature between about 5° C. (23° F.) and about −5° C. (41° F.). After cooling the tip 16 to the desired temperature, the tip is positioned into contact with the tissue. The radio frequency energy is then introduced into the tissue to ablate the underlying tissue cells. Cooling the tip 16 prevents tissue “charring” at the interface of the tip 16 and the tissue and allows higher amounts of radio frequency energy to pass into the tissue. The order of the steps involved in the ablation process using the catheter of the present invention may be other than described without departing from the scope of the present invention.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. A method of ablating tissue using a cooled high frequency ablation catheter, said method comprising:
supplying high frequency energy to the electrically conductive tip of the catheter;
providing a compressed fluid;
expanding the compressed fluid in an expansion chamber of the catheter to endothermically cool the fluid;
cooling the electrically conductive tip of the catheter using the expanding fluid;
exhausting the fluid from the expansion chamber;
bringing the tip into contact with a contact region of the tissue to deliver high frequency energy to the contact region and underlying tissue;
maintaining the contact region of the tissue at a temperature less than 50° C. (122° F.) to prevent charring of the contact region; and
heating by resistive heating the tissue underlying the contact region to a temperature of greater than 50° C. (122° F.) to ablate the underlying tissue.

2. The method of claim 1 wherein expanding the compressed fluid in an expansion chamber comprises cooling the fluid to a temperature below about 0° C. (32° F.).

3. The method of claim 1 wherein the tip is cooled to a temperature between about 5° C. (41° F.) and about −5° C. (23° F.) as the high frequency energy is being supplied to the tip.

4. The method of claim 1 wherein maintaining comprises selectively adjusting at least one of the amount of high frequency energy supplied to the tip and the amount of compressed fluid expanding in the expansion chamber.

5. The method of claim 4 wherein maintaining further comprises detecting the temperature adjacent the tip and automatically carrying out said adjusting step based on the detected temperature.

6. The method of claim 5 wherein providing a compressed fluid comprises providing one of compressed nitrous oxide gas and compressed carbon dioxide.

7. A method of ablating of tissue of a patient using a high frequency ablation catheter, said method comprising:
supplying an electrically conductive tip of the ablation catheter with high frequency energy;
contacting the contact region tissue of the patient with the tip of the ablation catheter;
directing the high frequency energy generally axially outward from the tip of the catheter through the contact region to ablate underlying tissue; and
cooling the tip of the ablation catheter to a temperature between about 5° C. (41° F.) and about −5° C. (23° F.)

8. The method of claim 7 wherein cooling is performed before contacting the tissue to adhere the tip to the tissue and make a conductive interface between the tissue and the tip.

9. A high frequency energy ablation catheter having a proximal end and a distal end, the catheter comprising:
a first passage having an inlet generally at the proximal end of the catheter and an outlet generally at the distal end of the catheter, said passage being adapted to deliver pressurized fluid from the inlet through the outlet;
an expansion valve disposed at the outlet of the first passage adapted to restrict flow out of the first passage to maintain pressure of the fluid within the first passage;
an expansion chamber in fluid communication with the expansion valve, the pressure within the expansion chamber being less than the pressure inside the first passage;
an electrically conductive tip adjacent the expansion valve at the distal end of the catheter, the tip being connected to a supply of high frequency energy for delivering the high frequency energy to tissue, the catheter being adapted to deliver the high frequency energy generally axially outward away from the distal and proximal ends of the catheter when the tip is in contact with tissue;
wherein pressurized fluid introduced into the first passage passes through the expansion valve and expands endothermically into the expansion chamber, thereby cooling the tip of the catheter.
